(19) United States
(12) Patent Application Publication
(10) Pub. No.: US 2024/0238165 A1
(43) Pub. Date: Jul. 18, 2024

(54) NASOGASTRIC TUBE PLACEMENT SYSTEM

(71) Applicant: Mohammad Nasser, Goodyear, AZ (US)

(72) Inventor: Mohammad Nasser, Goodyear, AZ (US)

(21) Appl. No.: 18/412,872

(22) Filed: Jan. 15, 2024

Related U.S. Application Data
(60) Provisional application No. 63/438,859, filed on Jan. 13, 2023.

(51) Int. Cl. A61J 15/00; A61M 25/01

(52) U.S. Cl. CPC A61J 15/0088; A61J 15/0003; A61J 2200/70; A61M 25/0127

(57) ABSTRACT

A nasogastric tube placement system includes a nasogastric tube with a plurality of magnets, a display covering with a plurality of lights and sensors, and a processor. The display covering has two layers defining a pocket where the sensors reside. The sensors detect the proximity to the magnets in the nasogastric tube and the processor controls the illumination of the lights based on the information from the sensors, showing the location of the nasogastric tube relative to the display covering. The system also includes a method of placing the nasogastric tube using the system, which involves placing the display covering on the patient, moving the nasogastric tube down the patient's esophagus, detecting the position of the magnets, and illuminating the lights to show the location of the tube.

### CROSS REFERENCE

This application claims benefit from currently pending U.S. Provisional Application No. 63/438,859 titled “Nasogastric Tube Placement System” and having a filing date of Jan. 13, 2024, and all of which is incorporated by reference herein.

### FIELD OF THE INVENTION

The present specification relates to a nasogastric tube placement and more specifically to a system that can be detect the nasogastric tube placement as it enters the patients throat and then into the stomach.

### BACKGROUND OF THE INVENTION

The placement of nasogastric (“NG”) tubes is a common medical procedure performed in various healthcare settings. These tubes are typically inserted through the nose, down the esophagus, and into the stomach to provide a means for feeding, medication administration, or gastric decompression. The NG tube is a long, thin, hallow tube that is passed through the patient's nostril down the throat and into the stomach. NG tubes are placed into the patient's stomach for providing hydration, medications, and feedings for those who are not able to safely take foods or liquids by mouth. Current procedures require the NG tube to be placed exactly in the stomach and proper insertion can be problematic. For example, the NG tube may enter the patient's lungs because of the proximity of the larynx to the esophagus and can puncture the patient's lung causing serious damage to the patient. However, incorrect placement of the NG tube can lead to serious complications, including pulmonary aspiration, pneumothorax, or even death. Therefore, accurate placement and confirmation of the NG tube's position is crucial.

Currently, there are a number of techniques in which the NG tube can be placed into a patient. The most common is blind placement where the NG tube is inserted transnasally and advanced to the cricopharyngeus. The tube is covered with some lubrication and numbing medication to help alleviate any discomfort. The patient's head is then flexed chin-to-chest to help narrow the trachea and open the esophagus. The tube is slowly advanced down into the stomach. Intragastric placement must be confirmed by auscultation of insufflated air in the epigastrium. If air cannot be auscultated, the tube is withdrawn into the nasopharynx and replaced. Intragastric positioning is usually indicated by light-colored secretions with minimal bile. Increased resistance usually suggests that coiling is occurring within the lungs. Other techniques require an X-ray of the patient's torso to see proper placement of the NG tube within the patient's stomach, if the NG tube is not properly placed then it is removed and reinserted for proper placement and an X-ray is taken again to show proper placement. Proper insertion of a NG tube can be time consuming and can severely damage the internal organs of the patient if not done correctly.

Other methods include the placement of the NG tube confirmed by radiographic imaging, which exposes the patient to radiation. This method requires the availability of radiology services and trained personnel, which may not be readily available in all healthcare settings. Furthermore, the need for repeated radiographic confirmation in cases of long-term NG tube usage can lead to increased healthcare costs and unnecessary radiation exposure for the patient.

Therefore, there is a need for a system that allows an individual to tract the NG tube and guide the user as it is placed within a patient. Such a system should provide real-time feedback on the position of the NG tube, be easy to use, and minimize patient discomfort and risk. It would also be beneficial if the system could communicate wirelessly with a portable computing device for ease of monitoring and data recording.

### BRIEF SUMMARY OF THE INVENTION

The present invention addresses the need for an improved nasogastric (“NG”) tube placement system. The system comprises an NG tube with a first and second end, where the NG tube has multiple magnets spaced from the first end towards the second end. A display covering has a first layer having a top surface and a bottom surface and the top surface is coupled with multiple lights. A second layer is coupled to the first layer to define a pocket between the two layers which houses multiple sensors that detect proximity to the magnets on the NG tube. A processor can be coupled to the lights and the sensors to selectively illuminate the lights based on information from the sensors, showing the location of the NG tube relative to the display covering.

In certain embodiments, the first and second layer of the display covering can be a flexible substrate. The sensors can be a magnetic sensor, a reed switch, or a hall effect sensor. The processor may further include a wireless communication module, such as Bluetooth Low Energy, which can send a signal to a portable computing device. The lights can be light emitting diodes, and the magnets can be a permanent magnet, neodymium iron boron magnet, samarium cobalt, ceramic magnet, or ferrite magnet.

The invention can also provide a method of placing an NG tube using the NG tube placement system. The method involves placing the display covering on the patient over the area where the NG tube is to be inserted, moving the NG tube down the patient's esophagus, detecting the position of the magnets based on proximity to the sensors, and selectively illuminating the lights based on information from the sensors to show the location of the NG tube relative to the display covering. Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims. The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for” and will also recite the word “function” (i.e., will state “means for performing the function of . . . , without also reciting in such phrases any structure, material or act in support of the function. Thus, even when the claims recite a “means for performing the function of molding a . . . , step for performing the function of molding a . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 is a top view of the nasogastric tube placement as shown in the prior art in accordance to one, or more embodiments;

FIG. 2 is a top view of the nasogastric tube placement system on a patient in accordance to one, or more embodiments;

FIG. 3 is a side view of the nasogastric tube placement system on a patient in accordance to one, or more embodiments;

FIG. 4 is an isometric view of the nasogastric tube placement sensing cover in accordance to one, or more embodiments;

FIG. 5 is a side view of the nasogastric placement tube in accordance to one, or more embodiments;

FIG. 6 is a cross-sectional view of the nasogastric tube placement sensing cover in accordance to one, or more embodiments; and

FIG. 7 is a back view of the nasogastric tube placement sensing cover in accordance to one, or more embodiments;

FIG. 8 is an exploded back view of the nasogastric tube placement sensing cover omitting the cover in accordance to one, or more embodiments; and

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

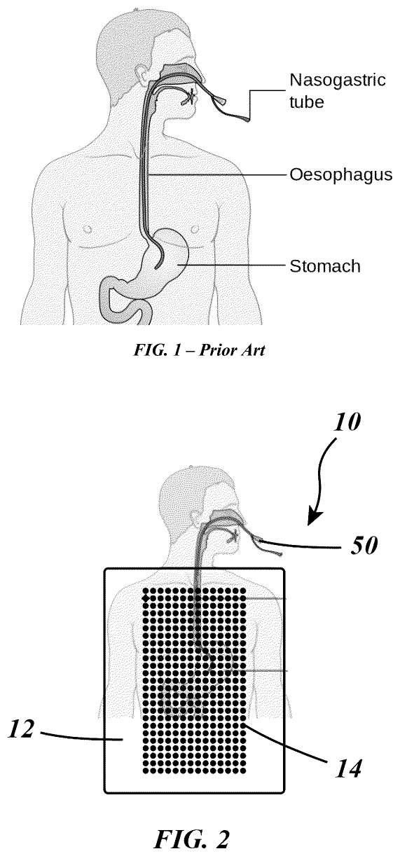

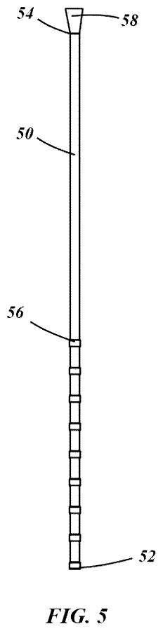

Referring initially to FIG. 1 a nasogastric (“NG”) tube is shown as used in the prior art. The NG tube is inserted into the patient's nose, down the oesophagus, and into the stomach. Currently, a user inserts the NG tube without being able to see where the NG tube is within the patient. Now referring to FIG. 2, a nasogastric tube placement system is shown generally at 10. The nasogastric tube placement system 10 comprises a NG tube 50 having a first end 52 and a second end 54. One or more magnets 56 are coupled to the NG tube near the first end 52 and extending toward the second end 54. A port 58 is coupled to the second end as shown in FIG. 5. The NG tube 50 can be manufactured from such as, for example, polyurethane, silicone, rubber, plastic or any other materials suitable for insertion into a patient.

The at least one magnet 56 can be spaced from the first end to the second end, or from the first end to the substantially the middle of the NG tube 50, or a substantially a quarter the way from the first end to the second end. The magnet 56 may wrap around the outer diameter of the tube, the inner diameter of the tube, can be flat magnets encapsulated by an outer sleeve, or the like. The magnet 56 may be, for example, a permanent magnet, a neodymium iron boron magnet, samarium cobalt, ceramic magnet, or ferrite magnet. The magnet 56 can have a radiused or chamfered edge to help alleviate any discomfort that may be felt by the patient as it is inserted into the patient's nose, mouth, esophagus and stomach. The magnet 56 can have a cover, sheath, or another tube wrapped around the magnet and NG tube 50 to keep the magnet in place or moving up the shaft of the NG tube, or the magnets can be molded into the NG tube. The magnets 52 can be such as, for example, a circular tube with substantially the same inner or outer diameter of the NG tube 50, or the magnets can be rectangular, square, or hexagonal placed on the top, bottom, or sides of the NG tube.

The magnets may go around the entire outer circumference of the NG tube or may be in a line on one side of the NG tube. The NG tube may be weighted to keep the magnets 52 oriented towards the surface of the patient's skin upon insertion of the tube 50.

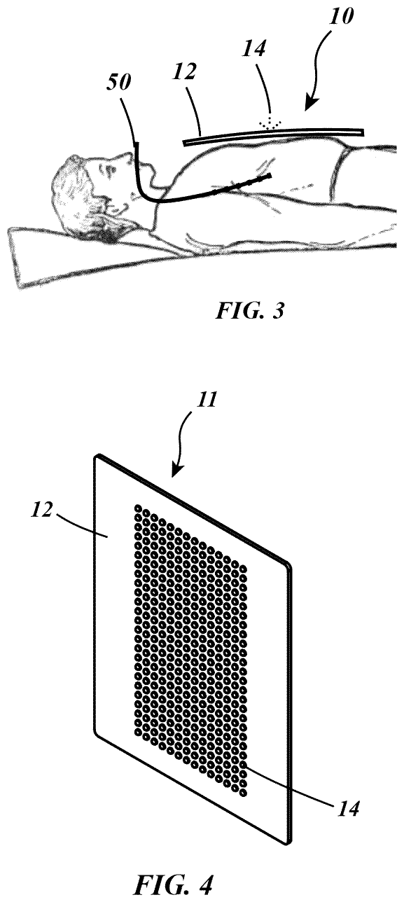

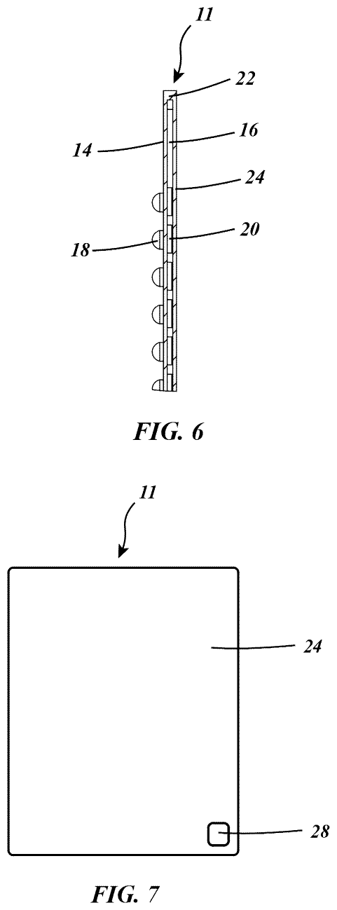

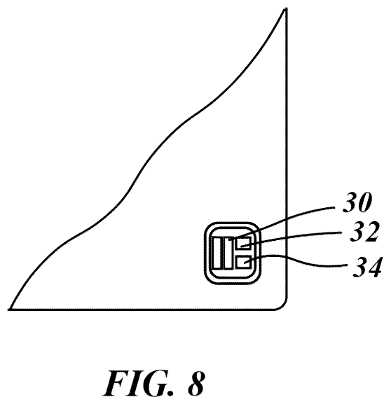

Referring to FIGS. 4, 6, and 7, the NG tube placement system 10 can further comprise a NG tube detection system 11 having a first layer 12 having top surface 14 and a bottom surface 15 wherein the top surface has a plurality of lights 18 coupled to the top surface. A second layer 24 may be coupled to the first layer at a periphery 22 to define a pocket 16 between the first layer 12 and the second layer 24. A plurality of sensors 20 may reside in the pocket and be coupled to the first layer 12. The thickness of the pocket can be such as, for example, between 0.005 inches to 0.250 inches, but more preferably between 0.010 inches to 0.200, but still more preferred 0.200 inches. In other embodiments the plurality of sensors 20 can be placed between the first layer 12 and the second 24 in a flexible substrate or wherein the plurality of lights 14 and the plurality of sensors are separated by a third layer (not shown) which can be an insulative layer keeping the lights and the sensors separated.

The first layer 12 and second layer 24 can be made from such as, for example, a flexible or solid substrate manufactured from acrylic, polycarbonate, LED panel sheet, polyimide, fabric, or the like. In other embodiments the first layer 12 and the second layer 24 can be different materials or can be interwoven into each other to create one layer after the lights 18 and sensors 20 are integrated within the layers.

The plurality of lights 18 can be evenly spaced over the top surface 14 of the first layer 12 or can be unevenly spaced on the top surface or it can be attached to the second layer 24. The plurality of lights 18 can be light emitting diodes (“LED”), florescent lights, flexible LEDs, or the like. The plurality of lights 18 can be attached by such as, for example, glue, sewn in, hook and loops, snaps, or the like. The plurality of lights 18 can be attached together to form one complete circuit but can operate independently of each other wherein the lights can turn on and off independently of each other and may be arranged as a pixel array. The plurality of lights 18 can sit flat against the first layer 12 or can be raised off the first layer's top surface or the lights can be integrated within the first layer 12.

The plurality of sensors 20 can be coupled to the first layer 12 wherein the sensors can be evenly or unevenly spaced in an array across the bottom surface. The plurality of sensors 20 can be such as, for example, reed switches, hall effect sensors, reed delays, magnetic sensors, tunnel magento resistance element, or the like. The sensors 20 can detect the magnets 56 on the NG tube 50 as it passes by each sensor and can show the location of the NG tube by lighting up the lights 18 as it passes by the sensor as shown in FIG. 3. As the magnets 56 pass by the plurality of sensors 20 each light 18 will illuminate showing the location of where the NG tube magnets 56 are respective to the sensors 20 thus showing the user the location of the NG tube 50 showing the location of the NG tube in real-time as shown in FIG. 3. The plurality of sensors 20 and lights can be coupled to a processor 32, wherein the processor can be configured to receive input from the sensors 20 indicating proximity to a magnet 56 on the NG tube 50. The processor 32 can receive the signal and selectively activate the lights 18 indicating to the user the location of the magnets on the NG tube 50 as it passes by or comes to rest on the sensor that is over the position in the patient's body.

The processor 32, sensors 20 and lights 18 can be connected to a power source 30 wherein the power source can be such as, for example, lithium, alkaline, carbon zinc, silver oxide, or the like. The power source 30 can be rechargeable and/or replaceable and in certain embodiments the power source can be connect directly to AC or DC power source by a power cord. In other embodiments the processor 32 can be connected to a wireless communication module 34 wherein the wireless communication module can be Bluetooth low energy, Wi-Fi, Bluetooth, or the like.

The wireless transmitter 34 can send a signal to a portable computing device wherein the portable computing device can have an application that can show the user the location of the NG tube 50 within the patient. The processor 32, power source 30 and wireless transmitter 34 can be contained between the first layer 14 and the third layer 24 by an enclosure (not shown) that can have a cover 28 attached to it.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claimed is:

1. A nasogastric (“NG”) tube placement system comprising:
a NG tube having a first end and a second end wherein the NG tube has a plurality of magnets spaced from the first end towards the second end;
a display covering having a first layer with top surface and a bottom surface wherein the top surface has a plurality of lights coupled to the top surface;
a second layer coupled to the first layer to define a pocket between the first layer and the second layer wherein the pocket has a thickness;
a plurality of sensors residing within the pocket and coupled to the first layer or the second layer that detect proximity to the plurality of magnets; and
a processor coupled to the lights and the sensors to selectively illuminate the plurality of lights based on information from the sensors to show the location of the NG tube relative to the display covering.

2. The NG tube placement system according to claim 1, wherein the first layer and second layer is a flexible substrate.

3. The NG tube placement system according to claim 1, wherein the sensor is at least one of a magnetic sensor, a reed switch, or a hall effect sensor.

4. The NG tube placement system according to claim 1, wherein the processor further comprises a wireless communication module wherein the wireless communication module is a Bluetooth Low Energy.

5. The NG tube placement system according to claim 4, wherein the wireless communication module can send a signal to a portable computing device.

6. The NG tube placement system according to claim 1, wherein the plurality of lights are light emitting diodes.

7. The NG tube placement system according to claim 1, wherein the magnet is a permanent magnet, neodymium iron boron magnet, samarium cobalt, ceramic magnet, or ferrite magnet.

8. A method of placing a NG tube using a NG tube placement system, the method comprising the acts of:
providing the NG tube placement system of claim 1;
placing the display covering on the patient over the area of the patient's body into which the NG tube is to be inserted;
moving the NG tube down the patient's esophagus;
detecting the position of the plurality of magnets based on proximity to the plurality of sensors; and
selectively illuminating the plurality of lights based on information from the sensors to show the location of the NG tube relative to the display covering.

9. The nasogastric tube placement system according to claim 1, wherein the lights are light emitting diodes.
